(19) United States
(12) Patent Application Publication
(10) Pub. No.: US 2023/0089770 A1
(43) Pub. Date: Mar. 23, 2023

(54) ANTIVIRAL CHIMERIC PEPTIDES

(71) Applicant: INNATE IMMUNITY LLC, Santa Fe, NM (US)

(72) Inventor: Goutam GUPTA, Santa Fe, NM (US)

(21) Appl. No.: 17/908,887

(22) PCT Filed: Mar. 2, 2021
(86) PCT No.: PCT/US2021/020383

§ 371 Date: Sep. 1, 2022

Related U.S. Application Data
(60) Provisional application No. 62/984,609, filed on Mar. 3, 2020.

(51) Int. Cl. C07K 14/415

(52) U.S. Cl. CPC C07K 14/415; C07K 2319/01

(57) ABSTRACT

The present disclosure relates generally to chimeric peptides composed of at least two domains connected by a linker. More specifically, the peptide domains may include an amphipathic helical domain and a targeting domain, which, in combination, allow the peptides to target and kill various viruses. The disclosed peptides may have a variety of beneficial agricultural properties and uses, for example, in the treatment of viruses that infect grapes, tobacco, tomatoes, citrus, and other commercially important crops.

### CROSS REFERENCE STATEMENT

This application claims priority under 35 U.S.C. § 119(e) to U.S. Provisional Application 62/984,609 filed Mar. 3, 2020, and the entire contents of this provisional application is incorporated herein by reference.

### FIELD OF INVENTION

The present disclosure relates generally to protein chimeras (i.e., chimeric peptides) composed of at least two domains connected by a linker. More specifically, the peptide/protein domains may include an amphipathic helical domain and a targeting domain, which, in combination, allow the peptides to target and kill various viruses. The disclosed peptides may have a variety of beneficial agricultural properties and uses, for example, in the treatment of viruses that infect grapes, tobacco, tomatoes, citrus, and other commercially important crops.

### BACKGROUND

The following discussion is merely provided to aid the reader in understanding the disclosure and is not admitted to describe or constitute prior art thereto.

Multiple agricultural crop viruses have been discovered over the years. For example, grapevine leafroll associated viruses (GLRaV) appear to be the most devastating of all viruses for grapes and are widespread worldwide. Several mealybug species are efficient vectors of GLRaV. Among 10 different viral subtypes, GLRaV-3 is the most infectious one, which can cause up to 40% losses in yield. Grapevine red blotch associated viruses (GRBaV) emerged only as recently as 2008 in California, and it is transmitted by leafhoppers. A recent survey of red blotch infected vines in the United States indicated about 15% losses in yield. Leafroll and red blotch virus infections disrupt immune defense pathways relating biotic and abiotic stress, carbohydrate metabolism, photosynthesis, respiration, electron transport, and hormonal balance. Disruptions in these pathways harm the berry quality because of irregular ripening, detrimentally impact plant growth, delay of sprouting, reduce tolerance to stresses, and even can even lead to death of chronically grapevines.

Similar agricultural viruses such as cucumber mosaic virus, tomato yellow leaf curl virus (TYLCV), African cassava mosaic virus (ACMV), plum pox virus (PPV), and tomato spotted wilt virus, to name a few, could benefit from improved antiviral treatments for agricultural diseases.

Accordingly, there is a need in the art for antiviral compounds that can be safely and efficiently provided to commercially valuable crops in order to protect these crops from disease or treat the disease. The present disclosure fulfills that need by providing combination chimeric proteins/peptides comprising a viral lysis domain and a viral targeting domain that can provide broad spectrum treatment and protect against agricultural viruses.

### SUMMARY

Described herein are novel chimeric peptides and methods of using the same to treat or prevent agricultural viral infections.

In one aspect, the disclosure provides an antiviral peptide comprising a recognition domain capable of binding to a virus coat protein and lytic domain comprising an amphipathic helical peptide sequence, wherein the recognition domain and the lytic domain are connected by a linker domain.

In some embodiments, the recognition domain or the lytic domain are derived from a plant. In some embodiments, the recognition domain and the lytic domain are derived from a plant. In some embodiments, the plant is a grape plant, citrus plant, tomato plant, or tobacco plant.

In some embodiments, the recognition domain is a subtilisin or a fragment or homolog thereof. In some embodiments, the recognition domain comprises SEQ ID NO: 1, 2, 3, 4, 5, 6, or 7. In some embodiments, the recognition domain comprises a subtilisin homolog comprising at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% of SEQ ID NO: 1, 2, 3, 4, 5, 6, or 7.

In some embodiments, the lytic domain comprises 8-50, 8-40, 8-30, 8-20, 8-15, 8-12, 8-11, 8-10, 10-50, 10-40, 10-30, 10-20, or 10-15 amino acids. In some embodiments, the lytic domain comprises the formula: (X¹ₙ X²ₒ)ₚ, wherein X¹ is a nonpolar amino acid residue, X² is a positively charged amino acid residue, n is 1-3, o is 1-3, and p is 1-3; (X¹ₙX²ₒ)ₚ, wherein X′ is a positively charged amino acid residue, X² is a nonpolar amino acid residue, n is 1-3, o is 1-3, and p is 1-3; X¹X²X³X⁴X⁵X⁶X⁷X⁸X⁹X¹⁰X¹¹, wherein X¹, X², X⁴, X⁵, X⁸, and X⁹ are nonpolar residues, wherein X³, X⁶, X¹⁰, and X¹¹ are positively charged residues, and wherein X⁷ is a positively charged residue or negatively charged residue; X¹X²X³X⁴X⁵X⁶X⁷X⁸X⁹X¹⁰X¹¹ wherein X², X⁵, X⁶, and X⁹ are positively charged residues, wherein X³, X⁴, X⁷, X⁸, X¹⁰ and X¹¹ are nonpolar residues, and wherein X¹ is a positively charged residue or negatively charged residue; or X¹X²X³X⁴X⁵X⁶X⁷X⁸X⁹X¹⁰X¹¹X¹², wherein X¹, X², X⁶, X⁸, and X¹² are positively charged residues, wherein X³ and X⁴ are nonpolar residues, wherein X⁵ is a polar, uncharged residue, X⁷ is selected from a nonpolar residue and positively charged residue, X⁹ is a nonpolar residue or negatively charged residue, X¹⁰ is a nonpolar residue or nonpolar, aromatic residue, and X¹¹ is a nonpolar residue or a polar, noncharged residue. In some embodiments, the lytic domain is a plant-derived amphipathic linear helical peptide (ALHP) or a fragment or homolog thereof. In some embodiments, the lytic domain comprises any one of SEQ ID NOs: 8-52 or 69-74. In some embodiments, the lytic domain comprises an ALHP homolog comprising at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% of any one of SEQ ID NOs: 8-52 or 69-74.

In some embodiments, the linker domain comprises 2-50, 3-25, or 4-12 amino acids. In some embodiments, the linker domain comprises 40-80% uncharged amino acid residues. In some embodiments, the linker domain comprises 10-60% positively charged amino acid residues. In some embodiments, the linker domain comprise repeats of 1-amino acids selected from the group consisting of Glycine-Serine, Arginine-Tryptophan, and Serine-Arginine-Aspartic Acid. In some embodiments, the linker domain comprises a mixture of polar and nonpolar amino acids in a ratio of 1:1, 1:2, or 2:1. In some embodiments, the linker domain comprises any one of SEQ ID NOs: 53-68. In some embodiments, the linker domain comprises a sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% of any one of SEQ ID NOs: 53-68.

In some embodiments: the recognition domain comprises the amino acid sequence of any one of SEQ ID NOs: 1-7 or a fragment thereof or a homolog possessing at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% amino acid sequence homology to any of the peptides disclosed in Table 1; the lytic domain comprises the amino acid sequence of any one of SEQ ID NOs: 8-52, SEQ ID NOs: 69-74, or a fragment thereof or a homolog possessing at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% amino acid sequence homology to any of the peptides disclosed in Table 2; and the linker domain comprises the amino acid sequence of any one of SEQ ID NOs: 53-68 or a fragment thereof or a homolog possessing at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% amino acid sequence homology to any of the peptides disclosed in Table 3.

In some embodiments: the recognition domain consists of the amino acid sequence of any one of SEQ ID NOs: 1-7 or a fragment thereof or a homolog possessing at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% amino acid sequence homology to any of the peptides disclosed in Table 1; the lytic domain consists of the amino acid sequence of any one of SEQ ID NOs: 8-52, SEQ ID NOs: 69-74, or a fragment thereof or a homolog possessing at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% amino acid sequence homology to any of the peptides disclosed in Table 2; and the linker domain consists of the amino acid sequence of any one of SEQ ID NOs: 53-68 or a fragment thereof or a homolog possessing at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% amino acid sequence homology to any of the peptides disclosed in Table 3.

In another aspect, the present disclosure provides a formulation comprising an antiviral peptide according to any one of the foregoing aspects or embodiments and an acceptable carrier or diluent. In some embodiments, the carrier is a solid. In some embodiments, the carrier is a liquid such as, for example, a spray or aerosol.

In another aspect, the present disclosure provides a method of treating or preventing a viral infection in a plant comprising, applying to a target area on or adjacent to a plant an effective amount of an antiviral peptide according to any one of the foregoing aspects or embodiments.

In some embodiments of the disclosed methods, the target area comprises a plant, the seed of a plant, or a portion of the plant. In some embodiments of the disclosed methods, the plant is a crop plant. In some embodiments of the disclosed methods, the plant is selected from the group consisting of grape, citrus, tobacco, and tomato.

In some embodiments of the disclosed methods, the target area is the soil in which a plant in growing, a field that will be planted, or a structure on which a plant is growing.

In some embodiments of the disclosed methods, the viral infection is caused by grape red blotch virus (GRBaV), grape leafroll (GLRaV), a xyloporosis virus, a tristeza virus, a psorosis virus, an excortis virus, tobacco mosaic virus, tomato mosaic virus, or tomato spotted wilt virus.

In some embodiments of the disclosed methods, applying comprises spraying the target with the antiviral peptide.

In another aspect, the present disclosure provides a method of treating or preventing a viral infection in a plant comprising, expressing within the plant an antiviral peptide according to any one of the foregoing aspects or embodiments.

In some embodiments of the disclosed methods, the plant is a crop plant. In some embodiments of the disclosed methods, the plant is selected from the group consisting of grape, citrus, tobacco, and tomato.

In some embodiments of the disclosed methods, the viral infection is caused by grape red blotch virus (GRBaV), grape leafroll (GLRaV),a xyloporosis virus, a tristeza virus, a psorosis virus, an excortis virus, tobacco mosaic virus, tomato mosaic virus, or tomato spotted wilt virus

In some embodiments of the disclosed methods, expression of the antiviral peptide does not require alteration of the plant genome. In some embodiments of the disclosed methods, the antiviral peptide is expressed by an expression vector or plasmid bound to a carbon nanotube (CNT) or by a non-infectious GLRaV-based delivery system.

The foregoing general description and following detailed description are exemplary and explanatory and are intended to provide further explanation of the disclosure as claimed. Other objects, advantages, and novel features will be readily apparent to those skilled in the art from the following brief description of the drawings and detailed description of the disclosure.

### BRIEF DESCRIPTION OF THE DRAWINGS

FIG. 1 shows schematic representations of the disclosed antiviral chimeric proteins comprising a recognition for domain targeting the viral coat proteins (e.g., a domain derived from Subtilisin) and a lytic domain for targeting the viral membrane.

FIG. 2 shows steps for designing the disclosed antiviral peptides. The sequences labeled 1-5 are antiviral peptides, and the underlined sequences in 1′-5′ represent the grape homolog of peptides 1-5. An additional N-terminal segment in 1′ is retained to favor the formation of a helix-turn-helix (HTH) structure, and the residues in green are replaced with the red to stabilize the HTH structure. In the sequences 2′ and 3′, an additional C-terminal segment in the homolog peptide is retained to strengthen HTH formation. In the sequences 4′ and 5′, two grape homologs are joined by a GPGR (SEQ ID NO: 55) peptide linker and a hydrophobic terminal residues are deleted to improve the solubility of the HTH peptides. Peptide 1=AVP0600 peptide with a minimum inhibitory concentration (MIC) 0.4 μM against HCV; Peptide 1′=grape homolog: hypothetical protein CK203_046801 [Vitis vinifera], Sequence ID: RVW89473.1. Peptide 2=AVP0588 peptide with MIC 0.51 μM against HCV; Peptide 2′=grape homolog: hypothetical protein CK203_076700 [Vitis vinifera], Sequence ID: RVW49563.1. Peptide 3=AVP0603 peptide with MIC 0.55 μM against HCV; Peptide 3′=grape homolog: putative ribonuclease H protein [Vitis vinifera], Sequence ID: RVX06017.1. 4=AVP0615 peptide with MIC 0.84 μM against HCV; Peptide 4′=grape homolog: putative disease resistance protein [Vitis vinifera], Sequence ID: RVW21616.1. Peptide 5tAVP0598 peptide with MIC 0.48 μM against HCV; Peptide 5′=grape homolog: E3 ubiquitin-protein ligase KEG [Vitis vinifera], Sequence ID: RVW93036.1.

FIG. 3 shows a schematic of yeast display method for selecting Subtilisin variants with improved activity and specificity toward the viral CPs.

FIG. 4 shows the design and use of non-GMO delivery systems. (A) CNT-plasmid DNA (encoding the antiviral chimera) can be attached to carbon nanotubes and transcribed in vivo without altering the genomic DNA of the organism expressing the plasmid; (B) GLRaV-7 with an exemplary chimera gene; (C) infiltration of CNT and GLRaV-7 for delivering the antiviral chimera. The volume and concentration of CNT and GLRaV infiltrated will be determined based upon the in planta expression of the delivered chimera by the two systems and the in vitro activity determined by the detached leaf assay.

### DETAILED DESCRIPTION

The present disclosure provides chimeric peptides with viral recognition and lysis domains that can clear viral infections and block the diseases in agricultural plants and crops, such as grapes. These antiviral chimeric peptides can be delivered transgenically (i.e., through recombinant in vivo expression) or via non-transgenic routes, which may include, but are not limited to, spraying the plant (e.g., the trunk, leaves, or fruit) with a suitable formulation of the chimeric peptides, delivery of carbon nanotube coated with the chimera genes that would be expressed in grape, or the expressions of the chimera genes in grape with the aid of a non-infectious viral vector. The disclosed antiviral chimeric peptides can be used to treat or prevent a variety of viral infections including, but not limited to, infections of grapes (e.g., GLRaV or GLBRaV), tobacco, tomatoes, citrus, and other commercially important crops.

The disclosed peptides function by improving/supplementing the intrinsic innate immunity in plants. This platform technology can be used for treating or preventing various viral-based agricultural diseases, such as grape leafroll and red blotch virus, which pose an imminent threat to the grape and wine industries in California and other grape growing regions around the world.

### I. Definitions

It is to be understood that methods are not limited to the particular embodiments described, and as such may vary. It is also to be understood that the terminology used herein is for the purpose of describing particular embodiments only, and is not intended to be limiting. The scope of the present technology will be limited only by the appended claims.

As used herein, certain terms may have the following defined meanings. As used in the specification and claims, the singular form “a,” “an” and “the” include singular and plural references unless the context clearly dictates otherwise. For example, the term “a peptide” includes a single peptide as well as a plurality of peptides, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting” of shall mean excluding more than trace elements of other ingredients for claimed compositions and substantial method steps. Embodiments defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

As used herein, “about” means plus or minus 10% as well as the specified number. For example, “about 10” should be understood as both “10” and “9-11.”

As used herein, “optional” or “optionally” means that the subsequently described event or circumstance may or may not occur, and that the description includes instances where said event or circumstance occurs and instances where it does not.

As used herein, “effective amount” means the amount of a peptide that provides the specific pharmacological effect for which the compound (e.g., the disclosed chimeric peptides) is administered to a plant in need of such treatment or protection, i.e. to reduce, ameliorate, eliminate, clear, or prevent a viral infection or one or more signs or symptoms of the viral infection. It is emphasized that an effective amount of a compound will not always be effective in treating the conditions/diseases described herein, even though such dosage is deemed to be an effective amount by those of skill in the art. The effective amount may vary based on the route of administration and formulation, the size of the plant, the virus being treated, and the severity of the infection, among other factors.

The terms “treatment” or “treating” as used herein with reference to plant or agricultural viral infections mean reducing, ameliorating, eliminating, or clearing a viral infection or one or more signs or symptoms of the viral infection.

The terms “prevent” or “protect” as used herein with reference to plant or agricultural viral infections mean blocking a viral infection from occurring in a plant or crop that is at risk of infection or has been exposed to a virus.

### II. Antiviral Chimeric Peptides

Provided herein are novel chimeric peptides that possess antiviral properties, among other beneficial activities. The disclosed peptides comprise at least a recognition domain, which targets one or more viral coat proteins (CP), and a lytic domain, which lyses the viral membrane, connected by a flexible linker (e.g., a peptide linker).

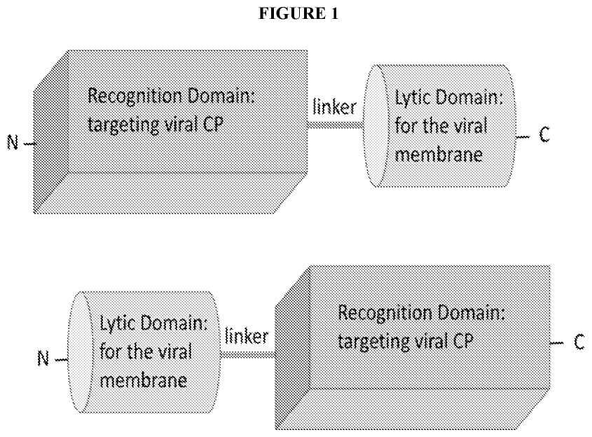

The lytic domain (e.g., a grade-derived antiviral helical peptide) and the recognition domain (e.g., a viral CP-cleaving subtilisin) can be joined by a flexible linker of about 3-25 amino acids to facilitate the synergy of membrane targeting and protease activities and consequently, which will lead to rapid clearance of the virus. FIG. 1 schematically shows the chimeras with the viral recognition and lysis domains, which can be either on the N or C terminus. Different chimeras may be constructed using different combinations of lytic peptides (e.g., those shown in FIG. 1) and different CP proteases (e.g., two grape Subtilisins CBI28067.3 and XP_002280942.1).

a. Recognition Domain

The recognition domain of the disclosed antiviral chimeric peptides generally comprises a protease or a fragment thereof that is capable of binding to one or more viral coat proteins (CPs).

In general, the recognition domain of the disclosed antiviral chimeric peptides will comprise a sequence of a subtilisin or a derivative or fragment thereof. Subtilisins are a family of proteases that belong to the class of subtilases, a group of serine proteases that initiate a nucleophilic attack on a peptide bond through a serine residue at the active site. Subtilisins typically have molecular weights of about 27 kDa. In some embodiments, the subtilisin is a subtilisin derived from a target plant (i.e., a plant intended for treatment or protection from a viral infection) or a homolog thereof. In general, the subtilisin is plant-derived, but in some embodiments bacterial subtilisin may be utilized as well.

For the purposes of the disclosed antiviral chimeric peptides, the subtilisin peptide or fragment thereof must be capable of binding a viral protein, such as a coat protein (CP) or viral envelope glycoprotein. Theoretical digestion maps of viral coat proteins can be obtained or determined from existing databased and putative recognition domains can be screened for binding affinity to the coat proteins.

For example, the viral coat proteins of GRBaV (ID=AMQ35562.1) and GLRaV-3 (ID=ABY87019.1) were analyzed, and it was determined that Proteinase K (or Subtilisin-like serine protease) was most efficient in digesting both of these viral CPs. Two grape Subtilisins (IDs=CBI28067.3 and XP 002280942.1) were identified as close homologs of initial viral CP-cleaving protease candidates. Those skilled in the art can readily identify homologs derived from other potential target plants, such as citrus, tobacco, and tomato plants. The binding and cleavage ability of any given subtilisin, subtilisin fragment, or subtilisin homolog can be validated by determining the experimental digestion of the recombinant subtilisins when introduced to recombinant viral CPs from a virus that may be the target of a treatment or prevention regimen. Recombinant CPs and subtilisins can be expressed in any suitable expression system, such as, an HEK cell, a CHO cell, or the like.

TABLE 1
Exemplary Recognition Domains

| SEQ | |
|---|---|
| ID | |
| NO: | Sequence |
| 1 | GLWEKGYTGAKVKMAIFDTGIRANHPHFRNIKERTNWTN |
| | EDTLNDNLGHGTFVAGVIAGQYDECLGFAPDTEIYAFRVF |
| | TDAQVSYTSWFLDAFNYAIATNMDVLNLSIGGPDYLDLPF |
| | VEKVWELTANNIIMVSAIGNDGPLYGTLNNPADQSDVIGV |
| | IDYGDHIASFSSRGMSTWEIPHGYGRVKPDVVAYGREIMG |
| | SSISANCKSLS |
| 2 | VQPHTTRSHEFLGLRRGSGAWTASNYGNGVIIGLVDSGIW |
| | PESASFKDEGMGKPPPRWKGACVADANFTSSMCNNKIIG |
| | ARYYNRGFLAKYPDETISMNSSRDSEGHGTHTSSTAAGAF |
| | VEGVSYFGYANGTAAGMAPRAWIAVYKAIWSGRIAQSD |
| | ALAAIDQAIEDGVDILSLSFSFGNNSLNLNPISIACFTAMEK |
| | GIFVAASAGNDGNAFGTLSNGEPWVTTVGAEMGTKPAP |
| | MVDIYSSRGPFIQCPNVLKPDILAPGTSVLAAWPSNTPVSD |
| | NFYHQWYSDFNVLSGTSMATAHVAGVAALVKAVHPNW |
| | SPAAIRSALMTTANTLDNT |
| 3 | GLWEKGYTGAKVKMAIFDTGIRANHPHFRNIKERTNWTN |
| | EDTLNDNLGHGTFVAGVIAGQYDECLGFAPDTEIYAFRVF |
| | TDAQVSYTSWFLDAFNYAIATNMDVLNLSIGGPDYLDLPF |
| | VEKVWELTANNIIMVSAIGNDGPLYGTLNNPADQSDVIGV |
| | IDYGDHIASFSSRGMSTWEIPHGYGRVKPDVVAYGREIMG |
| | SSISANCKSLSGTSVASPVVAGVVCLLVSVIPEHDRKNILN |
| | PASMKQALVEGAARLPDANMYEQGAGR |
| 4 | RGLWEKGYTGAKVKMAIFDTGIRANHPHFRNIKERTNWT |
| | NEDTLNDNLGHGTFVAGVIAGQYDECLGFAPDTEIYAFRV |
| | FTDAQVSYTSWFLDAFNYAIATNMDVLNLSIGGPDYLDLP |
| | FVEKVWELTANNIIMVSAIGNDGPLYGTLNNPADQSDVIG |
| | VIDYGDHIASFSSRGMSTWEIPHGYGRVKPDWAYGREIM |
| | GSSISANCKSLSGTSVASPVVAGVVCLLVSVIPEHDRKNIL |
| | NPASMKQALVEGAARLPDANMYEQGAGR |
| 5 | RAHQIHTTRTPHFLGLADNYGLWPNSDYADDVIIGVLDTG |
| | IWPEIRSFSDSGLSPVPNSWNGVCDTGPDFPASACNRKIIG |
| | ARAFFKGYEGALGRPMDESVESKSPRDTEGHGTHTASTA |
| | AGSVVQDASLFEFAKGEARGMAVKARIAAYKICWSLGCF |
| | DSDILAAMDQAVADGVDIISLSVGATGLAPRYDHDSIAIG |
| | AFGAMDHGVLVSCSAGNSGPDPLTAVNIAPWILTVGASTI |
| | DREFPADVVLGDGRIFGGVSIYSGDPLKDTNLPLVYAGDC |
| | GSRFCFTGKLNPSQVSGKIVICDRGGNARVEKGTAVKMA |
| | LGAGMILANTGDSGEELIADSHLLPATMVGQIAGDKIKEY |
| | VKSKAFPTATIVFRGTVIGTSPPAPKVAAFSSRGPNHLTPEI |
| | LKPDVIAPGVNILAGWTGSKAPTDLDVDPRRVEFNIISGTS |
| | MSCPHVSGLAALLRKAYPKWTPAAIKSALMTTAYNLDNS |
| 6 | KLHTTRSWDFLGMREKMKKRNPKAEINMVIGLLDTGIW |
| | MDCPSFKDKGYGPPPTKWKGKCSNSSGFTGCNNKVIGAK |
| | YYDLDHQPGMLGKDDILSPVDTDGHGTHTASTAAGIVVK |
| | NASLFGVGKGTARGGVPLARIAMYKVCWYTGCSDMNLL |
| | AGFDDAIADGVDVLSVSIGGTVGPFFEDPIAIGAFHAMRR |
| | GVLVSSSAGNDGPLEATVQNVAPWILTVGATGLDREFRS |
| | QVKLGNGMKASGVSVNTFSPRKKMYPLTSGTLASNSSGA |
| | YWGNVSACDWASLIPEEVKGKIVYCMGNRGQDFNIRDLG |
| | GIGTIMSLDEPTDIGFTFVIPSTFVTSEEGRKIDKYINSTKKA |
| | QAVIYKSKAFKIAAPFVSSFSSRGPQDLSPNILKPDIVAPGL |
| | DILAGYSKLAPISGDPEDRRFANFNILTGTSMSCPHVAAAA |
| | AYVKSFHPKWSPAAIKSALMTTATT |
| 7 | RYELHTTRTPEFLGLDKSADLFPESGSASEVIIGVLDTGIWP |
| | ESKSFDDTGLGPIPSSWKGECETGTNFTSSSCNRKLIGARF |
| | FSKGYEATLGPIDESKESKSPRDDDGHGTHTATTAAGSVV |
| | EGASLFGFAEGTARGMATRARIAAYKVCWIGGCFSTDILA |
| | ALDKAVEDNVNILSLSLGGGMSDYYRDSVAMGAFGAME |
| | KGILVSCSAGNSGPSPYSLSNVAPWITTVGAGTLDRDFPAF |
| | VSLGNGKNYSGVSLYRGDPLPGTLLPFVYAGNASNAPNG |
| | NLCMTNTLIPEKVAGKMVMCDRGVNPRVQKGSVVKAAG |
| | GIGMVLANTGTNGEELVADAHLLPATAVGQKSGDAIKSY |
| | LFSDHDATVTILFEGTKVGIQPSPVVAAFSSRGPNSITPDIL |
| | KPDLIAPGVNILAGWSGAVGPTGLPTDKRHVDFNIISGTS |
| | MSCPHISGLAGLLKAAHPEWSPAAIRSALMTTAYTN |

The list of subtilisin peptides provided in Table 1 is not intended to be limiting, and a skilled artisan would understand that other similar peptides or fragments can be derived from other plants and assessed for their ability to bind viral coat proteins. Additionally, in some embodiments the disclosed chimeric peptides may comprise homologous peptide to SEQ ID NOs: 1-7 that are derived from other plants.

In some embodiments, a subtilisin peptide or fragment thereof for use in the recognition domain of an antiviral chimeric peptide may possess about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% amino acid sequence homology to any of the peptides disclosed in Table 1.

b. Lytic Domain

The lytic domain of the disclosed antiviral chimeric peptides generally comprise an amphipathic helical peptide sequence that can be inserted into a viral membrane to lyse and kill the virus. The lytic domain may be isolated or derived from an endogenous plant protein or peptide. For example, many plants express amphipathic helical peptides that are capable of lysing viruses and bacteria as part of their innate immune system. Accordingly, the lytic domain of the disclosed antiviral chimeric peptides may be isolated or derived from a grape plant, a citrus plant, a tobacco plant, a tomato plant, an apple or pear plant, or any other commercially valuable crop or plant. In some embodiments, the lytic domain of the disclosed antiviral chimeric peptides is isolated or derived from the same type of plant that the peptide is intended to treat or protect (e.g., a lytic domain from a grape may be used to treat or prevent viral diseases of grape vines).

About 30 years ago, host amphipathic linear helical peptides (ALHPs) were discovered to possess antimicrobial activity against viral, bacterial, and fungal pathogens, and helical peptides with the highest activity or the lowest IC₅₀ (i.e., the concentration of the peptide at which the peptide reduces the plaque forming unit, pfu, to half on a log scale of the initial untreated condition) can be found in various protein databases, and once such a protein has been identified, a homolog from a plant of interest (e.g., a grape, citrus plant, tomato, etc.) can be determined as well.

In some embodiments, a suitable lytic domain may comprise a single plant-derived amphipathic helix or two helices engineered into a helix-turn-helix (HTH) format in which homologous or heterogeneous helices are connected by a peptide linker. Note that, we previously demonstrated that the HTH peptides possess higher antimicrobial activity than the constituent single helices because they are more efficient in membrane attachment, insertion, and rupture.

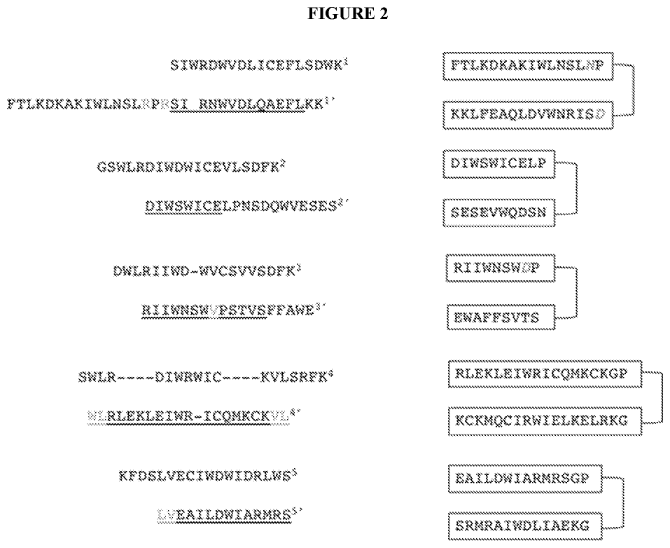

FIG. 2 outlines a representative process for identifying and preparing suitable helical peptides to serve as the lytic domain of the disclosed antiviral chimeric peptides. As shown in FIG. 2, the lytic domain may be optimized by strategically deleting or substituting particular amino acid residues to achieve a desired effect. Accordingly, in some embodiments, the lytic domain my comprise a wild-type amphipathic peptide sequence with 1, 2, 3, 4, or 5 or more substitution mutations and/or 1, 2, 3, 4, or 5 or more amino acid deletions at its N- or C-terminus.

In some embodiments, the lytic domain may consist of a single helical peptide, but in some embodiments, the lytic domain may comprise more than one helical peptide (e.g., 2, 3, 4, or more helical peptides) connected via a linker, such as a short peptide linker.

In some embodiments, the lytic domain comprises at least one helical peptide comprising 8-50, 8-40, 8-30, 8-20, 8-15, 8-12, 8-11, 8-10, 10-50, 10-40, 10-30, 10-20, or 10-15 amino acids. In some embodiments, the at least one helical peptide comprises 10-45, 10-35, 10-25, 10-20, 11-15, 11-28, 11-13, or 10-15 amino acids. In some embodiments, the at least one helical peptide comprises at least 7, 8, 9, 10, 11, 12, 13, 14, 15, 16, 17, 18, 19, or 20 or more amino acids. In some embodiments, the at least one helical peptide comprises 50, 45, 40, 37, 35, 34, 33, 32, 31, 30, 29, 28, 27, 26, 25, 20 or fewer amino acids.

In some embodiments, the lytic domain consists of at least one helical peptide comprising 8-50, 8-40, 8-30, 8-20, 8-15, 8-12, 8-11, 8-10, 10-50, 10-40, 10-30, 10-20, or 10-15 amino acids. In some embodiments, the at least one helical peptide consists of 10-45, 10-35, 10-25, 10-20, 11-15, 11-28, 11-13, or 10-15 amino acids. In some embodiments, the at least one helical peptide consists of at least 7, 8, 9, 10, 11, 12, 13, 14, 15, 16, 17, 18, 19, or 20 or more amino acids. In some embodiments, the at least one helical peptide consists of 50, 45, 40, 37, 35, 34, 33, 32, 31, 30, 29, 28, 27, 26, 25, 20 or fewer amino acids.

In some embodiments, one or more of the helical peptides of the lytic domain comprise an antiviral helix domain of a plant protein (e.g., an ALHP or peptide derived from an ALHP of a grape, tomato, tobacco, or citrus plant). In some embodiments, one or more of the helical peptides comprises an antimicrobial helix domain of a non-plant protein.

In some embodiments, the helical peptide is an amphipathic helix. In some embodiments, the amphipathic helix comprises alternating nonpolar amino acid residues and positively charged amino acid residues.

In some embodiments, the amphipathic helix comprises (X¹ₙX²ₒ)ₚ, wherein X¹ is a nonpolar amino acid residue, X² is a positively charged amino acid residue, n is 1-3, o is 1-3, and p is 1-3. In some embodiments, at least one X¹ is selected from L and I. In some embodiments, at least one X² is selected from R and K.

In some embodiments, the amphipathic helix comprises (X¹ₙX²ₒ)ₚ, wherein X¹ is a positively charged amino acid residue, X² is a nonpolar amino acid residue, n is 1-3, o is 1-3, and p is 1-3. In some embodiments, at least one X¹ is selected from R and K. In some embodiments, at least one X² is selected from L and I.

In some embodiments, a lytic domain may comprise the formula: X¹X²X³X⁴X⁵X⁶X⁷X⁸X⁹X¹⁰X¹¹, wherein X¹, X², X⁴, X⁵, X⁸, and X⁹ are nonpolar residues, wherein X³, X⁶, X¹⁰, and X¹¹ are positively charged residues, and wherein X⁷ is a positively charged residue or negatively charged residue.

In some embodiments, a lytic domain may comprise the formula: X¹X²X³X⁴X⁵X⁶X⁷X⁸X⁹X¹⁰X¹¹, wherein X², X⁵, X⁶, and X⁹ are positively charged residues, wherein X³, X⁴, X⁷, X⁸, X¹⁰ and X¹¹ are nonpolar residues, and wherein X¹ is a positively charged residue or negatively charged residue.

In some embodiments, a lytic domain may comprise the formula: X¹X²X³X⁴X⁵X⁶X⁷X⁸X⁹X¹⁰X¹¹, wherein X¹X², X⁶, X⁸, and X¹² are positively charged residues, wherein X³ and X⁴ are nonpolar residues, wherein X⁵ is a polar, uncharged residue, X⁷ is selected from a nonpolar residue and positively charged residue, X⁹ is a nonpolar residue or negatively charged residue, X¹⁰ is a nonpolar residue or nonpolar, aromatic residue, and X¹¹ is a nonpolar residue or a polar, noncharged residue.

In some embodiments, the nonpolar residue is selected from the group consisting of glycine (G), alanine (A), valine (V), leucine (L), methionine (M), and isoleucine (I). In some embodiments, the nonpolar residue is selected from the group consisting of A, L, and I. In some embodiments, the nonpolar amino acid is selected from the group consisting of L and I.

In some embodiments, the positively charged amino acid residue is selected from lysine (K), arginine (R), and histidine (H). In some embodiments, the positively charged amino acid residue is selected from K and R.

In some embodiments, any of the helical peptides disclosed herein may comprise an amino acid sequence consisting of 0-4 amino acid residues selected from the group consisting of polar uncharged residues, negatively charged residues, and nonpolar aromatic residues. In some embodiments, the helix domain comprises 4, 3, 2, or 1 or fewer polar uncharged residues, negatively charged residues, and/or nonpolar aromatic residues.

In some embodiments, the polar uncharged residues are selected from the group consisting of serine (S), threonine (T), cysteine (C), proline (P), asparagine (N), and glutamine (Q).

In some embodiments, the negatively charged residues are selected from the group consisting of aspartate (D) and glutamate (E).

In some embodiments, the nonpolar aromatic residues are selected from the group consisting of phenylalanine (F), tyrosine (Y), and tryptophan (W).

In some embodiments, when the lytic domain comprises 2 or more helical peptides, the one or more additional helix peptides are identical to the first helix, while in some embodiments, the one or more additional helix peptides are different than the first helix. In some embodiments, the first helix and second helix differ by 1-4 amino acid residues. In some embodiments, the first helix and second helix differ by 1, 2, 3, 4, 5 amino acid residues. In some embodiments, the first helix and second helix differ by 5, 4, 3, 2, or 1 or fewer amino acid residues. In some embodiments, the second helix consists of an amino acid sequence that is the reverse of the amino acid sequence of the first helix. In some embodiments, the first helix and the second helix are the same length. In some embodiments, at least two helical peptides are of the same length. In some embodiments, the first helix and the second helix are different lengths. In some embodiments, at least two helical peptides are different lengths. In some embodiments at least two helical peptides differ by 1, 2, 3, 4, or 5 amino acids in length.

In some embodiments, a helical peptide of the lytic domain comprises a mixture of positively charged amino acid residues and nonpolar amino acid residues. In some embodiments, the ratio of positively charged amino acid residues to nonpolar amino acid residues is 0.7:1, 0.75:1, 0.8:1, 0.9:1, or 1:1. In some embodiments, the ratio of positively charged amino acid residues to nonpolar amino acid residues is 1.1:1, 1.2:1, 1.3:1, 1.4:1 and 15:1.

Helical peptides that are suitable for use as the lytic domain of the disclosed antiviral chimeric peptides can be derived from commercially valuable agricultural crops including, but not limited to, grape, citrus, tobacco, and tomato plants.

Table 2 provides exemplary helical peptides that have been derived from grape and citrus plants.

TABLE 2
Exemplary Helical Peptides for a Lytic Domain

| SEQ | |
|---|---|
| ID NO: | Sequence |
| 8 | LYKKLSKKLL |
| 9 | LIKLIKKILKK |
| 10 | LIRLIRRILRR |
| 11 | KRIVQRIKDFLR |
| 12 | KRLVQRLKDFLR |
| 13 | KRLIQRKRLIQR |
| 14 | LYKKLSKKLLGPGRLYKKLSKKLL |
| 15 | LIKLIKKILKKGPGRKKLIKKILKIL |
| 16 | LIRLIRRILRRGPGRRRLIRRILRIL |
| 17 | LIRLLRRILRRGPGRRRLIRRLLRIL |
| 18 | LIRLLREILRRGPGRERLIRRLLRIL |
| 19 | LIRLILRILRRGPGRRRLIRLILRIL |
| 20 | LIRLISRILRRGPGRRRLIRSILRIL |
| 21 | ALYLKDFKSSKSLDVSALADLKHLKRL |
| 22 | KRIVQRIKDFLRGPGRKRIVQRIKDFLR |
| 23 | KRLVQRLKDFLRGPGRKRLVQRLKDFLR |
| 24 | KRLIQRKRLIQRGPGRKRLIQRKRLIQR |
| 25 | LIKLIKKILKKGPGRKKLIKKILKILGPGR |
| 26 | KKLIKKILKILGPGRKKLIKEILKILGPGRKKLIKKILKIL |
| 27 | ALYLKDFKSSKSLDVSALADLKHLKRLGPGRALYLKDFK |
| | SSKSLDVSALADLKHLKRL |
| 28 | GRLIKLIKKILKKGPGRKKLIKKILKILGP |
| 29 | LIKLCKKILKKGPGRKKLIKKCLKIL |
| 30 | LIKKILKILKK |
| 31 | KKLAKEILKAL |
| 32 | KKLIKKILKIL-(NHCH3) |
| 33 | RRLIRRILRIL |
| 34 | RRLIRRILRIL-(NCH3) |
| 35 | LIKLIKKILKKGPGRKKLIKKILKIL |
| 36 | LIRLIRRILRRGPGRRRLIRRILRIL |
| 37 | LIRLLRRILRRGPGRRRLIRRLLRIL |
| 38 | LLIKLIKKILKKGPGRKKLIKKILKILL |
| 39 | KRIVQRIKDFLRGPGRKRIVQRIKDFLR |
| 40 | KRLIQRKRLIQRGPGRKRLIQRKRLIQR |
| 41 | KLIKLIKKILKKGPGRKKLIKKILKILK |
| 42 | KLIRLIREILRRGPGRRRLIREILRILK |
| 43 | KEIVRRIKEFLRGPGRKEIVRRIKEFLR |
| 44 | KEIVRRIEKFLRGPGRKRIVERIEKFLR |
| 45 | HPLIKLIKKILKKGPGRKKLIKKILKILGH |
| 46 | ELLRRLLASLRRHDLLRGPGRELLRLLASLRRHDLLR |
| 47 | EALRSRLEKRIYILYRDTPVVKSSSRQREELLRISLRELE |
| 48 | RLLEKRLRRELERELRKQGPGRRLLEKRLRRELERELRKQ |
| 49 | RKQLRELIERLLERIRKLGPGRREQLERLIERLERLIEKR |
| 50 | KKLIKKILKILGPGRKKLIKEILKILGPGRKKLIKKILKIL |
| 51 | GRLIKLIKKILKKGPGRKKLIKKILKILGP |
| 52 | LIKLCKKILKKGPGRKKLIKKCLKIL |
| 69 | CRTRGCGCHLCRMLSQFTGG |
| 70 | CDMASSGCDVCECRVLLPEE |
| 71 | NQGRHFCGGALIHARFVMTAASCFQ |
| 72 | GDYLHFCMGALIHARTVMSGFDSDA |
| 73 | SIWRDWVDLICEFLSDWK |
| 74 | FTLKDKAKIWLNSLNPDSIRNWVDLQAEFLKK |

The list of helical peptides provided in Table 2 is not intended to be limiting, and a skilled artisan would understand that other similar helical peptides can be derived from other plants and assessed for their ability to lyse viral membranes. Additionally, in some embodiments the disclosed chimeric peptides may comprise homologous peptide to SEQ ID NOs: 8-52 or 69-74 that are derived from other plants.

In some embodiments, a helical peptide for use in the lytic domain of an antiviral chimeric peptide may possess about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% amino acid sequence homology to any of the helical peptides disclosed in Table 2.

Additional or alternatively, red blotch (GRBaV) proteins or other viral proteins or fragments, variants, or derivatives thereof may be combined with a recognition domain and a linker disclosed herein to prepare a functional antiviral peptide. For example, the peptide sequences QFAFHGNSFPSGGFRLYLNAF (SEQ ID NO: 75), GVNKKPQESLPHSRA (SEQ ID NO: 76), ILDRFPTGTDPSVSD (SEQ ID NO: 77), VMKKRSRQRKQRRRRRTTGRSSAIRRRAR (SEQ ID NO: 78), or DNSGSMIETLGGSGQFAFHGNSFPSGGFRLYLNA (SEQ ID NO: 79) may be combined with any of the disclosed recognition domains and linkers disclosed herein. In some embodiments, a peptide for use in the lytic domain of an antiviral chimeric peptide may possess about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% amino acid sequence homology to any of SEQ ID NOs: 75-79.

c. Linker Domain

The flexible linker that connects the recognition domain and the lytic domain is generally 2-50 amino acids in length, and should ideally facilitate synergy between the protease activity of the recognition domain and the membrane-lysing activity of the lytic domain. For instance, in some embodiments, the linker may be 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length. In some embodiments, the linker may be 3-15, 3-12, 4-10, 4-9, 4-8, 5-15, 5-10, 5-25, 10-20, 10-30, 10-40, or 15-25 amino acids in length.

In some embodiments, the linker comprises 40-80% uncharged amino acid residues. In some embodiments, a helix domain disclosed herein comprises the linker comprises 35%, 40%, 45%, 50%, 55%, 60%, 65%, 70%, 75%, or 80% uncharged amino acid residues.

In some embodiments, the linker comprises 10-60% positively charged amino acid residues. In some embodiments, the linker comprises at least 5%, 6%, 7%, 8%, 9%, 10%, 11%, 12%, 13%, 14%, or 15% positively charged amino acid residues. In some embodiments, the linker comprises 60%, 55%, 50%, 45%, 40%, 35%, 30%, or fewer positively charged amino acid residues.

In some embodiments, the linker may comprise repeats of 1-3 or 2-3 amino acids such as, Glycine-Serine, Arginine-Tryptophan, Serine-Arginine-Aspartic Acid, etc. The linker may comprise a mixture of polar and nonpolar amino acids, for example, an alternating pattern of polar and nonpolar amino acids, and the ratio of polar to polar amino acids may be 1:1, 1:2, or 2:1.

Table 3 below provides exemplary linkers that are suitable for incorporation into the disclosed antiviral chimeric peptides.

TABLE 3
Exemplary Linkers

| SEQ | |
|---|---|
| ID NO: | Sequence |
| 53 | AAA |
| 54 | GGGSSGGGSG |
| 55 | GPGR |
| 56 | RDTPVVKS |
| 57 | AKDGIPAPTNYHKKHRAPVSCTGPAKM |
| 58 | GSTAPPA |
| 59 | RANATTLPKYYQNSRHPVSCTDPSK |
| 60 | RW |
| 61 | SRD |
| 62 | GSTAPPA |
| 63 | GSTAPPAGSTAPPA |
| 64 | QASHTCVCEFNCAPL |
| 65 | ARKKASIPNYYNSNLQPPVF CSDQSKM |
| 66 | YEQGAGRGSTAPPA |
| 67 | GSTA |
| 68 | GGGSGGGTDGR |

The list of linker peptides provided in Table 3 is not intended to be limiting, and a skilled artisan would understand that other similar peptides can be used to operably connect the lytic domain and the recognition domain.

In some embodiments, a linker peptide may possess about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% amino acid sequence homology to any of the linker peptides disclosed in Table 3.

In some embodiments, the disclosed antiviral chimeric peptides may comprise a recognition domain and a lytic domain connected by a linker domain, wherein:
the recognition domain comprises the amino acid sequence of any one of SEQ ID NOs: 1-7 or a fragment thereof or a homolog possessing at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% amino acid sequence homology to any of the peptides disclosed in Table 1;
the lytic domain comprises the amino acid sequence of any one of SEQ ID NOs: 8-52, SEQ ID NOs: 69-74, or a fragment thereof or a homolog possessing at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% amino acid sequence homology to any of the peptides disclosed in Table 2; and
the linker domain comprises the amino acid sequence of any one of SEQ ID NOs: 53-68 or a fragment thereof or a homolog possessing at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% amino acid sequence homology to any of the peptides disclosed in Table 3.

In some embodiments, the disclosed antiviral chimeric peptides may consist of a recognition domain and a lytic domain connected by a linker domain, wherein:
the recognition domain comprises the amino acid sequence of any one of SEQ ID NOs: 1-7 or a fragment thereof or a homolog possessing at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% amino acid sequence homology to any of the peptides disclosed in Table 1;
the lytic domain comprises the amino acid sequence of any one of SEQ ID NOs: 8-52, SEQ ID NOs: 69-74, or a fragment thereof or a homolog possessing at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% amino acid sequence homology to any of the peptides disclosed in Table 2; and
the linker domain comprises the amino acid sequence of any one of SEQ ID NOs: 53-68 or a fragment thereof or a homolog possessing at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% amino acid sequence homology to any of the peptides disclosed in Table 3.

In some embodiments, the disclosed antiviral chimeric peptides may consist of a recognition domain and a lytic domain connected by a linker domain, wherein:
the recognition domain consists of the amino acid sequence of any one of SEQ ID NOs: 1-7 or a fragment thereof or a homolog possessing at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% amino acid sequence homology to any of the peptides disclosed in Table 1;
the lytic domain consists of the amino acid sequence of any one of SEQ ID NOs: 8-52, SEQ ID NOs: 69-74, or a fragment thereof or a homolog possessing at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% amino acid sequence homology to any of the peptides disclosed in Table 2; and
the linker domain consists of the amino acid sequence of any one of SEQ ID NOs: 53-68 or a fragment thereof or a homolog possessing at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% amino acid sequence homology to any of the peptides disclosed in Table 3.

The disclosed antiviral chimeric peptides may be produced by known means in the art such as, for example, recombinant expression in a suitable host cell. In some embodiments, the disclosed peptides may be expressed by human embryonic kidney (HEK) cells or Chinese hamster ovary (CHO) cells or any other available cell expression system. Alternatively, the peptides may be expressed by or in the plant for which protection or treatment is desired.

### III. Formulations of the Disclosed Antiviral Chimeric Peptides

Formulations suitable for use in the methods described herein can be formulated with one or more of the disclosed antiviral chimeric peptides and an acceptable carrier or diluent. The content of the antiviral chimeric peptides within a formulated composition may be from about 0.01 to about 95%. The antiviral chimeric peptides may be formulated into various types of compositions, including but not limited to an oil solution, emulsifiable concentrate, wettable powder, flowable (aqueous suspension or aqueous emulsion), granule, dust and so on, by mixing with solid carrier, liquid carrier, or gaseous carrier and optionally surfactant, the other formulation additive.

Non-limiting examples of solid carriers that can be used in a formulation comprising the disclosed antiviral chimeric peptides include inorganic carriers such as clays (e.g., kaolin clay, diatomaceous earth, synthetic hydrated silicon oxide, bentonite, Fubasami clay, acid clay), talc, ceramics, sericite, quartz and calcium carbonate. Examples of the liquid carrier include water, alcohols (e.g., methanol, ethanol, higher alcohols), ketones (e.g., acetone, methyl ethyl ketone), aromatic hydrocarbons (e.g., benzene, toluene, xylene, ethylbenzene, methylnaphthalene), aliphatic hydrocarbons (e.g., hexane, cyclohexane, kerosene, gas oil), esters (ethyl acetate, butyl acetate), nitrites (e.g., acetonitrile, isobutyronitrile), ethers (e.g. diisopropyl ether, dioxane), acid amides (e.g., N,N-dimethylformamide, N,N-dimethylacetamide), halogenated hydrocarbons (e.g., dichloromethane, trichloroethane, carbon tetrachloride), dimethyl sulfoxide and vegetable oils (e.g., soybean oil, cottonseed oil). Examples of the liquefied gaseous carrier include fluorocarbon, fluorohydrocarbon, LPG (liquefied petroleum gas), dimethyl ether and carbon dioxide.

Non-limiting examples of the surfactant optionally used in the disclosed formulations can include alkyl sulfate salts, alkylsulfonate salts, alkylarylsulfonate salts, alkyl aryl ethers, polyoxyethylenealkyl aryl ethers, polyethylene glycol ethers, polyhydric alcohol esters and sugar alcohol derivatives.

The other formulation auxiliaries are exemplified by sticking agents, dispersants, and stabilizers. Non-limiting examples of sticking agents and dispersants include casein, gelatin, polysaccharides (e.g., starch powder, gum arabic, cellulose derivatives, alginic acid), lignin derivatives, bentonite, sugars and synthetic water-soluble polymers (e.g., polyvinyl alcohol, polyvinylpyrrolidone, polyacrylic acids). Non-limiting examples of stabilizer include phenol type antioxidants such as BHT (2,6-di-tert-butyl-4-methyphenol) and BHA (mixture of 2-tert-butyl-4-methoxyphenol and 3-tert-butyl-4-methoxyphenol), amine type antioxidants such as diphenylamine, organic sulfur type antioxidants such as 2-mercaptobenzimidazole, PAP (acid isopropyl phosphate), vegetable oils, mineral oils, surfactants, fatty acids and esters of fatty acid.

Flowable formulations (aqueous suspension or aqueous emulsion) may comprise one or more of the disclosed antiviral chimeric peptides, a dispersant, a suspension assistant (for example, protective colloid or a compound giving thixotropy), suitable auxiliaries (for example, antifoamer, rust preventive agent, stabilizer, developing agent, penetrating assistant, antifreezing agent, bactericide, fungicide, etc.) and water. Non-limiting examples of a protective colloid include gelatin, casein, gums, cellulose ethers and polyvinyl alcohol, and examples of the compound giving thixotropy include bentonite, aluminum magnesium silicate, xanthan gum and polyacrylic acids. Use of an oil, which can, in some instance, solubilize a disclosed antiviral chimeric peptides, in place of water can give suspension-in-oil formulation.

The formulations of emulsifiable concentrate, wettable powder, flowable and so on obtained above may be diluted with water or another suitable vehicle, and applied at 0.1 to 10000 ppm of the concentration of the antiviral chimeric peptides. The formulations of oil solution, granule, dust and so on are may be applied to an intended plant, seed, trunk, or leaf directly as they are.

In some embodiments, a mixture of one or more of the disclosed antiviral chimeric peptides or a liquid formulation thereof and a propellant can be charged into a pressure container with a spray nozzle to afford an aerosol of the disclosed antiviral chimeric peptides. Non-limiting examples of the propellant for aerosols include propane, butane, isobutane, dimethyl ether, methyl ethyl ether and methylal.

In some embodiments, rather than applying the disclosed antiviral chimeric peptides to a specific plant, the antiviral chimeric peptides may be applied (e.g., sprayed or otherwise dispersed) in a general target area where it is desirable to prevent the spread of a particular virus or treat a diseased population of plants. The target area may be, for example, a site of a known infection or a field where crops are being grown.

The application amount and concentration of the disclosed antiviral chimeric peptides that should be applied to a given plant or target area can be suitably designed according to the type of the formulations, time, place, and method of application, kind of target plant, the type of virus, and the type of use desired (e.g., treatment or prevention).

### IV. Methods of Using the Disclosed Antiviral Chimeric Peptides

The disclosed antiviral chimeric peptides are useful for a variety of agricultural applications. In one aspect, the present disclosure provides methods of using one or more of the disclosed antiviral chimeric peptides to treat or prevent viral infection in a plant. The one or more antiviral chimeric peptides may be the same (i.e., comprise the same recognition, lytic, and linker domains) or different (i.e., comprise one or more distinct domain between the recognition, lytic, and linker domains). The one or more peptides may be administered to a plant, a target site on a plant, or a target site near a plant (e.g., the dirt around the roots or a fence, arbor, or other structure on which a vine is growing or intended to grow) or were a plant is intended to be cultivated (e.g., a field prior to planting a crop). Additionally or alternatively, the one or more peptides may be expressed by the target plant that is intended for treatment or protection by introducing a gene or expression vector encoding the one or more peptides into the target plant.

The disclosed antiviral chimeric peptides improve on the innate antimicrobial defense of a target plant by utilizing a plant-derived recognition domain to recognize and bind to a virus and a plant-derived lytic domain to penetrate the viral membrane and destroy the virus via lysis. The viral envelope is generally composed of phospholipids, proteins, and glycoproteins, which encompass the viral genome or capsid. Many viruses possess an envelope that comprises coat proteins that may be bound by a recognition domain and membranes that may be lysed by a lytic domain, and given the non-specific nature of this mechanism of action, the disclosed antiviral chimeric peptides are believed to possess broad spectrum antiviral activity.

Accordingly, the disclosed methods of treating or preventing a viral infection in a target plant can be applied to treating or preventing, for example, grape infections like red blotch (GRBaV) or leafroll (GLRaV); citrus infections like xyloporosis, tristeza, psorosis, or excortis; tobacco infections like tobacco mosaic virus; tomato infections like tomato mosaic virus or tomato spotted wilt virus; and the like. In some embodiments, the disclosed methods can be used to treat or prevent other viral diseases including, but not limited to, cucumber mosaic virus, tomato yellow leaf curl virus (TYLCV), African cassava mosaic virus (ACMV), plum pox virus (PPV), and tomato spotted wilt virus.

In some embodiments, the disclosed methods can be used to treat or prevent red blotch or leafroll in grape plants. Red blotch (GRBaV) and leafroll (GLRaV) are, respectively, envelope and filamentous viruses that infect grape plants, and these viruses derive their envelope components from the host grape. Although, the viral envelope/membrane is different from the host grape membrane in terms of the presence of covalently attached and non-covalently associated protein complexity and dynamics of the membrane lipid components, appropriate lytic peptides can be selected to target GRBaV and GLRaV-3.

Many commercially valuable crops and agricultural plants are effected by viral infections and these infections can result in severe economic loss. The disclosed methods may be used to treat or prevent viral infections (e.g., GRBaV and/or GLRaV) in any plant including, but not limited to, grape, citrus, tobacco, tomato, cucumber, plum, and other fruits, vegetables, legumes, nuts, etc.

For the purposes of the disclosed methods, an antiviral chimeric peptide may be applied to a plant, seed, or portion of a plant three or more times a day, twice a day, or once a day. In some embodiments, the antiviral chimeric peptide may be applied once a day, once every other day, three times a week, twice a week, once a week, once every other week, once every three weeks, once a month, once every other month, once every three months, once every four months, once every five months, once every six months, or less frequently. In such embodiments, the antiviral chimeric peptide may be applied to a plant, either sequentially or concurrently, with one or more additional insecticides, larvicides, fungicides, antibiotics, anti-microbials, herbicides, or arthropod repellents.

For the purposes of the disclosed methods, the antiviral chimeric peptides be applied to the intended plant, seed, or portion of a plant in any appropriate form, such as in a spray, aerosol, liquid, gel, powder, or solid form. The antiviral chimeric peptides may be formulated and applied to a plant, seed, or portion of a plant as solids, liquids, or gases (e.g., using a vapor delivery system). Alternatively or additionally, one or more expression vectors encoding the disclosed antiviral chimeric peptides may be introduced into the plant via transgenic or non-transgenic techniques such that the desired antiviral chimeric peptides are expressed by the target plant to prevent or treat a viral infection.

For example, the antiviral chimeric peptides described herein can be applied via a number of formulation types, including isolated antiviral chimeric peptides, which may further be coupled with dustable powders (DP), soluble powders (SP), water soluble granules (SG), water dispersible granules (WG), wettable powders (WP), granules (GR) (slow or fast release), soluble concentrates (SL), oil miscible liquids (OL), ultra-low volume liquids (UL), emulsifiable concentrates (EC), dispersible concentrates (DC), emulsions (both oil in water (EW) and water in oil (EO)), micro-emulsions (ME), suspension concentrates (SC), oil-based suspension concentrate (OD), aerosols, fogging/smoke formulations, capsule suspensions (CS) and seed/plant treatment formulations.

In some embodiments, delivery of the antiviral chimeric peptides to plants can be via different routes. The antiviral chimeric peptides can be suitably administered as an aerosol, for example by spraying onto leaves or other plant material. The particles can also be administered by injection, for example directly into a plant, such as into the stem. In certain embodiments the antiviral chimeric peptides are administered to the roots. This can be achieved by spraying or watering plant roots with compositions. In some embodiments, the antiviral chimeric peptides are introduced into the xylem or phloem, for example by injection or being included in a water supply feeding the xylem or phloem. Application to the stems or leaves of the plant can be performed by spraying or other direct application to the desired area of the plant; however, any method known in the art can be used. A solution or vehicle containing the antiviral chimeric peptides at a dosage of active ingredient can be applied with a sprayer to the stems or leaves until runoff to ensure complete coverage, and repeat three or four times in a growing season. The concentrations, volumes and repeat treatments may change depending on the plant, route of administration, and virus being treated or prevented.

Additional embodiments of the invention include a polynucleotide comprising a nucleic acid sequence that may encode one or more of the antiviral chimeric peptides described herein. For example, some embodiments may include a polynucleotide comprising a nucleic acid sequence that encodes one of more of SEQ ID NOs: 1-79. Such sequences may further be operably linked to a promotor to generate an expression vectors and further introduced to a plant, preferably a commercially valuable crop, including but not limited to, a grape plant, citrus plant, tobacco plant, tomato plant, or any other crop that is susceptible to viral infection. In this embodiment, such transformed plant or plant cell may produce the antiviral chimeric peptide(s) in vivo. Such a transformed plant may exhibit enhanced resistance to any number of agricultural viruses. In some embodiments, a transformed plant may exhibit decreased viral loads and/or decreased symptoms or progression of given viral disease (e.g., GRBaV or GLRaV-3). Methods, systems and techniques of stable and transient plant transformation, such as Agrobacterium tumefaciens-mediated transformation, carbon nanotube (CNT)-based transformation, and the like are known in the art and included within the scope of the present disclosure.

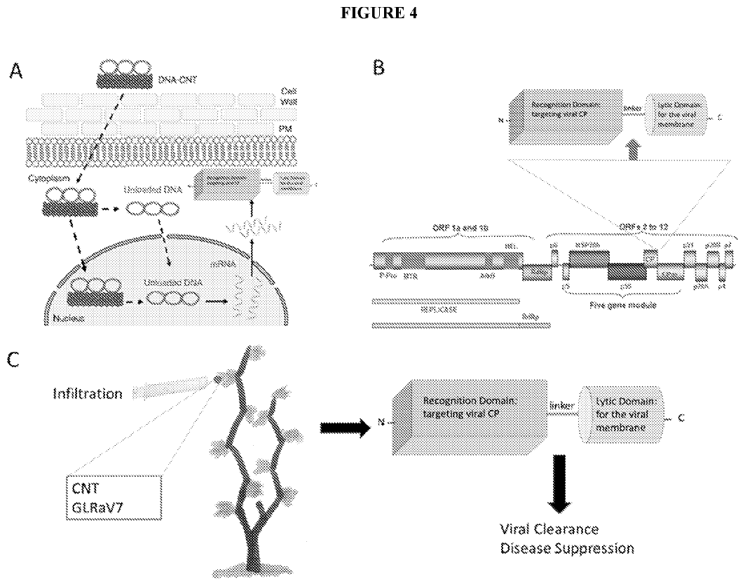

Briefly, transformation methods for expressing the antiviral chimeric peptides in vivo within with plants targeted for treatment or protection may include, but are not limited to, in planta methods the delivery of the antiviral chimera genes that do not involve alteration of the plant (e.g., grape) genome (i.e., non-GMO routes), such as carbon nanotube (CNT) (FIG. 4A) and non-infectious GLRaV-7 (FIG. 4B) based delivery systems.

For the purposes of a CNT-based approach, single wall COOH-CNT can be linked to positively charged poly-ethylen-imine (PEI), on which high copy pUC plasmid DNA encoding one or more of the antiviral chimeric peptides can be bound by electrostatic interactions. The gene encoding the antiviral chimeric peptide contain an N-terminal secretion signal and upstream 2X35S CaMV and downstream NOS terminator. CNT-PEI-DNA may be about 10 to about 20 nm in length to allow the nanoparticle to penetrate the extracellular matrix and enter the plant cell (see FIG. 4). CNT-PEI-DNA particles can be introduced into a target plant by various routes, such as injection into the leaves by needleless syringes. Following expression of the transformed antiviral chimeric peptide(s) within the target plant, the protein chimera will exert antiviral activity.

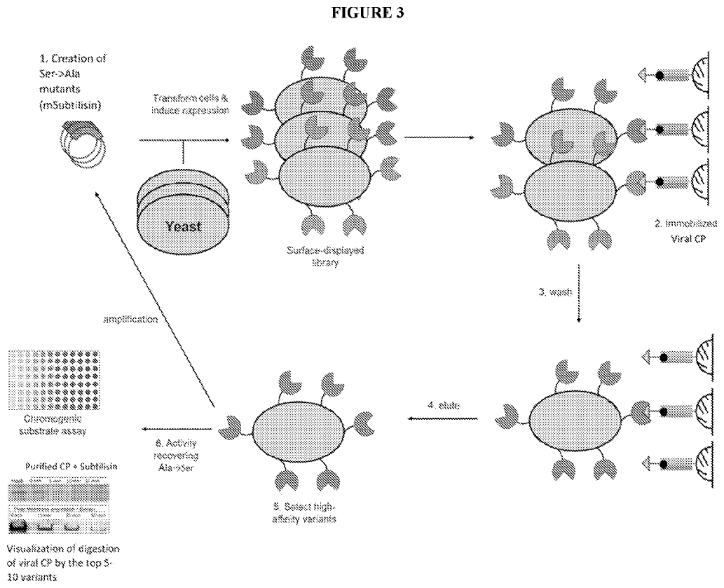

For the purposes of a non-infectious GLRaV-7 route, agrobacterium containing a binary vector harboring GLRaV-7 with one or more genes encoding one or more of the disclosed antiviral chimeric peptides can be infiltrated in the leaves. This allows a sustained in planta delivery and transport of the antiviral chimeric peptide throughout the plant or in a targeted tissue of the plant. The viral gene cassette and the chimera gene can be driven by, for example, a 2X35S CaMV promoter and NOS terminator. Design of Subtilisins highly specific to GLRaV-3 CP, as described in FIG. 3, will leave the GLRaV-7 unaffected.

In some embodiments, the plant to which the disclosed antiviral chimeric peptides are applied may be a crop plant, fruit, vegetable, legumes, nut, cotton, tobacco, etc.

The following examples are given to illustrate the present invention. It should be understood, however, that the invention is not to be limited to the specific conditions or details described in these examples.

### EXAMPLES

### Example 1—Production, Efficacy Testing, and Optimizing Antiviral Chimeric Peptides Derived from Grapes

Different chimeras are expressed in and purified (in mg quantities) from human cells using established protocols. Briefly, the DNA encoding various chimeras is synthesized from Genscript (codon optimized for HEK293F, pUC57, Kan+). The DNA encoding the chimeras is cloned in pcDNA3.1+/C-DYK (BamHI/EcoRI) site, which includes a TEV (Tobacco Etch Virus) protease cleavable FLAG tag. The recombinant clone is selected by restriction digestion and DNA sequencing. The plasmid is prepared from overnight culture (Maxiprep Kit) and will be used for transfecting HEK293F cells (30 μm) following manufacturer's instructions. Following transfection, the cell suspension is collected every day for 5 days and protein expression is monitored by western blot analysis to determine the optimum condition for expression. The recombinant protein is expressed under the optimum condition and will be affinity purified using FLAG tag resin. The FLAG tag from the purified protein is removed by TEV cleavage. The viral CPs, GRBaV (ID=AMQ35562.1) and GLRaV-3 (ID=ABY87019.1), are also expressed in and purified from HEK cells.

The antiviral efficacy of different chimeras is determined using a detached leaf assay. For this, leaves are collected from leafroll and red blotch infected grapevines in Norther/Central California. Six top chimeras with lytic peptides with high antiviral activity and subtilisins with high CP cleavage activity are initially chosen for analysis. For each virus infection, 35 leaves are tested in each biological replicate: 5 leaves each for the treatment of 6 chimeras and 5 leaves for treatment with water (control). Three biological replicates with 35 leaves in each replicate are analyzed for the presence of GRBaV or GLRaV-3 DNA specific loci by qPCR using virus-specific primers as reported for GRBaV [13] and GLRaV-3. A reference DNA, a grape gene encoding the endochitinase PR4-like protein (LOC100266390: XM_002274383.3) is used to demonstrate that the reference DNA level is not affected by treatment. The level of clearance in the treated sample is measured relative to the untreated sample. This will allow ranking of the chimeras in terms of their relative activity on GRBaV or GLRaV-3.

The antiviral specificity and activity of the top-ranked chimera is further improved by yeast display. See FIG. 3, in which different steps are shown to select subtilisins with improved specificity toward a leafroll or red blotch virus CP. First, a library of plasmids with mSubtilisin genes (with Ser -> Ala substitution at the catalytic Asp, His, Ser triad) is cloned. mSubtilisin genes encode proteins that will be devoid of catalytic activity but may retain binding to the viral CP. Second, yeast are transformed with the plasmids to express mSubtilisin non-binders (grey) and binders (green/purple) on the surface of yeast. Third, CP-immobilized beads are used to isolate yeast variant with high affinity mSubtilisin binders (purple) on the surface. Fourth, Ala-> Ser may be altered at the catalytic site to recover and measure the protease activity using a chromogenic synthetic substrate. Fifth, the steps are repeated to further improve the protease activity of subtilisin on CP. Finally, after several rounds of evolution, 5-10 subtilisin variants are selected with high activity and specificity toward the viral CP.

High affinity and specificity subtilisin may replace the original grape subtilisin in the top-ranked chimera.

### Example 2—Large-Scale Production of High Activity/Specificity Anti-Viral Chimeras and a Small-Scale Field Trial

Field efficacy testing requires>100 gms of the anti-viral chimeras. Therefore, transgenic tobacco is used to extract and purify the chimeras. For this, tobacco seeds (cv Barley) are surface sterilized using 1% sodium hypochlorite for 25 minutes followed by washing in water. Surface sterilized seeds are then be allowed to germinate in germination medium in magenta boxes (MS+15% sucrose, 0.3% phytagel). After germination, several millimeters long leaves are obtained, which can be transformed. Overnight culture of Agrobacterium strain (AGL1) harboring the gene encoding (CaMV35S: Chimera: TEV/GST tag: NOS) in a binary vector (pBI121) is adjusted to 0.5 (OD 600) and is used for inoculation of leaf discs (MS+30% sucrose, 100 μM acetosyringone and 10 μM β-Me). The infected leaves are incubated at 25 C in dark for 3 days. Following cocultivation, the leaf discs are placed in selection medium containing kanamycin (100 mg/L) and cefortaxime (250 mg/L) with subculture every 2 weeks. After induction, shoots are transferred to rooting medium in magenta boxed and allowed to grow until the plant is established. Transgenic plants are screened by genomic PCR using construct specific primers and the positive lines are transferred to the greenhouse for generation of seeds (Sun et al., 2006). Seeds from T1 lines are germinated and used for production and purification of the recombinant protein. The expression of the chimera mRNA is analyzed from different tissues (leaf, root) and at different stages by qPCR. The samples are collected at the point of maximum expression and the protein is isolated. The leaf tissues (20 gm) are crushed to fine powder in liquid nitrogen in a buffer containing 20 mM Tris-HCl(pH7.5), 150 mM NaCl, 20 mM KCl, 10 mM MgCl₂, 1 mM PMSF and protease inhibitor cocktail tablets. The suspension is filtered through triple layer of cheese cloth and the filtered solution is centrifuged at 12,000 rpm for 15 mins. The supernatant is collected and concentrated and then loaded into a glutathione sepharose column for GST-specific purification, after which, the chimera is obtained after TEV digestion and liberation of GST.

The isolated antiviral chimeras are in small-scale field efficacy studies for red blotch and leafroll disease treatment in grapevines. For each grapevine viral disease, 4 blocks of vines are chosen: block 1=10 uninfected vines; block 2=10 infected untreated (spread with 1% Pentra bark) vines; block 3=20 infected vines treated with anti-viral chimera 1; block 4=20 infected vines treated with two antiviral chimeras. Blocks 3 and 4 are sprayed on the bark with (100 ml, 20 μM) chimera 1 and chimera 2 in 1% Pentra bark on day 1, 5, and 10 and viral load is monitored at day 3, 7, 15, 30, 60, and 90. The symptoms are monitored for 6 months after chimera treatment. This trial requires about 3 gm of each chimera.

Two additional field efficacy studies (in the Northern and Central California) are conducted using CNT and GLRaV-7 systems to express the best antiviral chimera proteins against GRBaV and GLRaV-3 in grapevines.

For each trial, 6 blocks of infected grapevines are selected. Block 1 (10 grapevines) is infiltrated with CNT without any DNA coating (untreated control). Block 2 (10 grapevines) is infiltrated with Agrobacterium containing an empty binary vector (untreated control). Blocks 3 and 4 (distant from each other; 20 grapevines each) are infiltrated with CNT-Chimera on day 1, 31, and 61. Blocks 5 and 6 (distant from each other; 20 grapevines each) are infiltrated with Agrobacterium expressing GLRaV-7/chimera on day 1, 31, and 61. Leaf infiltration is performed at 3 different parts of each grapevine. The leaf samples are analyzed by qPCR every week for 3 months to measure the viral load using the method described under Objective 2. Symptoms are monitored for 6 months.

All patents and publications mentioned in the specification are indicative of the levels of those of ordinary skill in the art to which the disclosure pertains. All patents and publications are herein incorporated by reference to the same extent as if each individual publication was specifically and individually indicated to be incorporated by reference.

Further, one skilled in the art readily appreciates that the present disclosure is well adapted to carry out the objects and obtain the ends and advantages mentioned, as well as those inherent therein. Modifications therein and other uses will occur to those skilled in the art. These modifications are encompassed within the spirit of the disclosure and are defined by the scope of the claims, which set forth non-limiting embodiments of the disclosure.

1. An antiviral peptide comprising a recognition domain capable of binding to a virus coat protein and lytic domain comprising an amphipathic helical peptide sequence, wherein the recognition domain and the lytic domain are connected by a linker domain.

2. The antiviral peptide of claim 1, wherein the recognition domain or the lytic domain are derived from a plant.

3. The antiviral peptide of claim 1, wherein the recognition domain and the lytic domain are derived from a plant.

4. The antiviral peptide of claim 2, wherein the plant is a grape plant, citrus plant, tomato plant, or tobacco plant.

5. The antiviral peptide of claim 1, wherein the recognition domain is a subtilisin or a fragment or homolog thereof.

6. The antiviral peptide of claim 1, wherein the recognition domain comprises SEQ ID NO: 1, 2, 3, 4, 5, 6, or 7.

7. The antiviral peptide of claim 1, wherein the recognition domain comprises a subtilisin homolog comprising at least about 80% identity to any one of SEQ ID NOs: 1, 2, 3, 4, 5, 6, or 7.

8. The antiviral peptide of claim 1, wherein the lytic domain comprises 8-50 amino acids.

9. The antiviral peptide of claim 1, wherein the lytic domain comprises the formula:
a) (X1n X2o)p, wherein X¹ is a nonpolar amino acid residue, X² is a positively charged amino acid residue, n is 1-3, o is 1-3, and p is 1-3;
b) (X1n X2o)p, wherein X¹ is a positively charged amino acid residue, X² is a nonpolar amino acid residue, n is 1-3, o is 1-3, and p is 1-3;
c) X1X2X3X4X5X6X7X8X9X10X11, wherein X1, X2, X4, X5, X8, and X9 are nonpolar residues, wherein X3, X6, X10, and X11 are positively charged residues, and wherein X7 is a positively charged residue or negatively charged residue;
d) X1X2X3X4X5X6X7X8X9X10X11, wherein X2, X5, X6, and X9 are positively charged residues, wherein X3, X4, X7, X8, X10 and X11 are nonpolar residues, and wherein X1 is a positively charged residue or negatively charged residue; or
e) X1X2X3X4X5X6X7X8X9X10X12, wherein X1, X2, X6, X8, and X12 are positively charged residues, wherein X3 and X4 are nonpolar residues, wherein X5 is a polar, uncharged residue, X7 is selected from a nonpolar residue and positively charged residue, X9 is a nonpolar residue or negatively charged residue, X10 is a nonpolar residue or nonpolar, aromatic residue, and X11 is a nonpolar residue or a polar, noncharged residue.

10. The antiviral peptide of claim 1, wherein the lytic domain is a plant-derived amphipathic linear helical peptide (ALHP) or a fragment or homolog thereof.

11. The antiviral peptide of claim 1, wherein the lytic domain comprises any one of SEQ ID NOs: 8-52 or 69-74.

12. The antiviral peptide of claim 1, wherein the lytic domain comprises an ALHP homolog comprising at least about 80% identity to any one of any one of SEQ ID NOs: 8-52 or 69-74.

13. The antiviral peptide on claim 1, wherein the linker domain comprises 2-50, 3-25, or 4-12 amino acids.

14. The antiviral peptide of claim 1, wherein the linker domain comprises 40-80% uncharged amino acid residues.

15. The antiviral peptide of claim 1, wherein the linker domain comprises 10-60% positively charged amino acid residues.

16. The antiviral peptide of claim 1, wherein the linker domain comprise repeats of 1-amino acids selected from the group consisting of Glycine-Serine, Arginine-Tryptophan, and Serine-Arginine-Aspartic Acid.

17. The antiviral peptide of claim 1, wherein the linker domain comprises a mixture of polar and nonpolar amino acids in a ratio of 1:1, 1:2, or 2:1.

18. The antiviral peptide of claim 1, wherein the linker domain comprises any one of SEQ ID NOs: 53-68.

19. The antiviral peptide of claim 1, wherein the linker domain comprises a sequence comprising at least about 80% identity to any one of SEQ ID NOs: 53-68.

20. The antiviral peptide of claim 1, wherein:
a) the recognition domain comprises the amino acid sequence of any one of SEQ ID NOs: 1-7;
b) the lytic domain comprises the amino acid sequence of any one of SEQ ID NOs: 8-52 or 69-74; and
c) the linker domain comprises the amino acid sequence of any one of SEQ ID NOs: 53-68.

21. The antiviral peptide of claim 1, wherein:
a) the recognition domain consists of the amino acid sequence of any one of SEQ ID NOs: 1-7;
b) the lytic domain consists of the amino acid sequence of any one of SEQ ID NOs: 8-52 or 69-74; and
c) the linker domain consists of the amino acid sequence of any one of SEQ ID NOs: 53-68.

22. A formulation comprising an antiviral peptide claim 1 and an acceptable carrier or diluent.

23. The formulation of claim 22, wherein the carrier is a solid.

24. The formulation of claim 22, wherein the carrier is a liquid.

25. The formulation of claim 24, wherein the liquid is a spray or aerosol.

26. A method of treating or preventing a viral infection in a plant comprising, applying to a target area on or adjacent to a plant an effective amount of an antiviral peptide of claim 1.

27-30. (canceled)

31. The method of claim 26, wherein the viral infection is caused by grape red blotch virus (GRBaV), grape leafroll (GLRaV),a xyloporosis virus, a tristeza virus, a psorosis virus, an excortis virus, tobacco mosaic virus, tomato mosaic virus, or tomato spotted wilt virus.

32-35. (canceled)

36. The method of claim 31, wherein the viral infection is caused by grape red blotch virus (GRBaV), grape leafroll (GLRaV),a xyloporosis virus, a tristeza virus, a psorosis virus, an excortis virus, tobacco mosaic virus, tomato mosaic virus, or tomato spotted wilt virus

37-38. (canceled)


SEQUENCE LISTING (94 sequences)

SEQ ID NO: 1 - protein, 210 aa, Unknown
  source: /note=“Description of Unknown recognition domain sequence”
GLWEKGYTGAKVKMAIFDTGIRANHPHFRNIKERTNWTNEDTLNDNLGHGTFVAGVIAGQYDECLGFAPDTEIYAFRVFTDAQVSYTSWFLDAFNYAIATNMDVLNLSIGGPDYLDLPFVEKVWELTANNIIMVSAIGNDGPLYGTLNNPADQSDVIGVIDYGDHIASFSSRGMSTWEIPHGYGRVKPDVVAYGREIMGSSISANCKSLS

SEQ ID NO: 2 - protein, 334 aa, Unknown
  source: /note=“Description of Unknown recognition domain sequence”
VQPHTTRSHEFLGLRRGSGAWTASNYGNGVIIGLVDSGIWPESASFKDEGMGKPPPRWKGACVADANFTSSMCNNKIIGARYYNRGFLAKYPDETISMNSSRDSEGHGTHTSSTAAGAFVEGVSYFGYANGTAAGMAPRAWIAVYKAIWSGRIAQSDALAAIDQAIEDGVDILSLSFSFGNNSLNLNPISIACFTAMEKGIFVAASAGNDGNAFGTLSNGEPWVTTVGAEMGTKPAPMVDIYSSRGPFIQCPNVLKPDILAPGTSVLAAWPSNTPVSDNFYHQWYSDFNVLSGTSMATAHVAGVAALVKAVHPNWSPAAIRSALMTTANTLDNT

SEQ ID NO: 3 - protein, 267 aa, Unknown
  source: /note=“Description of Unknown recognition domain sequence”
GLWEKGYTGAKVKMAIFDTGIRANHPHFRNIKERTNWTNEDTLNDNLGHGTFVAGVIAGQYDECLGFAPDTEIYAFRVFTDAQVSYTSWFLDAFNYAIATNMDVLNLSIGGPDYLDLPFVEKVWELTANNIIMVSAIGNDGPLYGTLNNPADQSDVIGVIDYGDHIASFSSRGMSTWEIPHGYGRVKPDVVAYGREIMGSSISANCKSLSGTSVASPVVAGVVCLLVSVIPEHDRKNILNPASMKQALVEGAARLPDANMYEQGAGR

SEQ ID NO: 4 - protein, 268 aa, Unknown
  source: /note=“Description of Unknown recognition domain sequence”
RGLWEKGYTGAKVKMAIFDTGIRANHPHFRNIKERTNWTNEDTLNDNLGHGTFVAGVIAGQYDECLGFAPDTEIYAFRVFTDAQVSYTSWFLDAFNYAIATNMDVLNLSIGGPDYLDLPFVEKVWELTANNIIMVSAIGNDGPLYGTLNNPADQSDVIGVIDYGDHIASFSSRGMSTWEIPHGYGRVKPDVVAYGREIMGSSISANCKSLSGTSVASPVVAGVVCLLVSVIPEHDRKNILNPASMKQALVEGAARLPDANMYEQGAGR

SEQ ID NO: 5 - protein, 480 aa, Unknown
  source: /note=“Description of Unknown recognition domain sequence”
RAHQIHTTRTPHFLGLADNYGLWPNSDYADDVIIGVLDTGIWPEIRSFSDSGLSPVPNSWNGVCDTGPDFPASACNRKIIGARAFFKGYEGALGRPMDESVESKSPRDTEGHGTHTASTAAGSVVQDASLFEFAKGEARGMAVKARIAAYKICWSLGCFDSDILAAMDQAVADGVDIISLSVGATGLAPRYDHDSIAIGAFGAMDHGVLVSCSAGNSGPDPLTAVNIAPWILTVGASTIDREFPADVVLGDGRIFGGVSIYSGDPLKDTNLPLVYAGDCGSRFCFTGKLNPSQVSGKIVICDRGGNARVEKGTAVKMALGAGMILANTGDSGEELIADSHLLPATMVGQIAGDKIKEYVKSKAFPTATIVFRGTVIGTSPPAPKVAAFSSRGPNHLTPEILKPDVIAPGVNILAGWTGSKAPTDLDVDPRRVEFNIISGTSMSCPHVSGLAALLRKAYPKWTPAAIKSALMTTAYNLDNS

SEQ ID NO: 6 - protein, 462 aa, Unknown
  source: /note=“Description of Unknown recognition domain sequence”
KLHTTRSWDFLGMREKMKKRNPKAEINMVIGLLDTGIWMDCPSFKDKGYGPPPTKWKGKCSNSSGFTGCNNKVIGAKYYDLDHQPGMLGKDDILSPVDTDGHGTHTASTAAGIVVKNASLFGVGKGTARGGVPLARIAMYKVCWYTGCSDMNLLAGFDDAIADGVDVLSVSIGGTVGPFFEDPIAIGAFHAMRRGVLVSSSAGNDGPLEATVQNVAPWILTVGATGLDREFRSQVKLGNGMKASGVSVNTFSPRKKMYPLTSGTLASNSSGAYWGNVSACDWASLIPEEVKGKIVYCMGNRGQDFNIRDLGGIGTIMSLDEPTDIGFTFVIPSTFVTSEEGRKIDKYINSTKKAQAVIYKSKAFKIAAPFVSSFSSRGPQDLSPNILKPDIVAPGLDILAGYSKLAPISGDPEDRRFANFNILTGTSMSCPHVAAAAAYVKSFHPKWSPAAIKSALMTTATT

SEQ ID NO: 7 - protein, 476 aa, Unknown
  source: /note=“Description of Unknown recognition domain sequence”
RYELHTTRTPEFLGLDKSADLFPESGSASEVIIGVLDTGIWPESKSFDDTGLGPIPSSWKGECETGTNFTSSSCNRKLIGARFFSKGYEATLGPIDESKESKSPRDDDGHGTHTATTAAGSVVEGASLFGFAEGTARGMATRARIAAYKVCWIGGCFSTDILAALDKAVEDNVNILSLSLGGGMSDYYRDSVAMGAFGAMEKGILVSCSAGNSGPSPYSLSNVAPWITTVGAGTLDRDFPAFVSLGNGKNYSGVSLYRGDPLPGTLLPFVYAGNASNAPNGNLCMTNTLIPEKVAGKMVMCDRGVNPRVQKGSVVKAAGGIGMVLANTGTNGEELVADAHLLPATAVGQKSGDAIKSYLFSDHDATVTILFEGTKVGIQPSPVVAAFSSRGPNSITPDILKPDLIAPGVNILAGWSGAVGPTGLPTDKRHVDFNIISGTSMSCPHISGLAGLLKAAHPEWSPAAIRSALMTTAYTN

SEQ ID NO: 8 - protein, 10 aa, Unknown
  source: /note=“Description of Unknown helical peptide”
LYKKLSKKLL

SEQ ID NO: 9 - protein, 11 aa, Unknown
  source: /note=“Description of Unknown helical peptide”
LIKLIKKILKK

SEQ ID NO: 10 - protein, 11 aa, Unknown
  source: /note=“Description of Unknown helical peptide”
LIRLIRRILRR

SEQ ID NO: 11 - protein, 12 aa, Unknown
  source: /note=“Description of Unknown helical peptide”
KRIVQRIKDFLR

SEQ ID NO: 12 - protein, 12 aa, Unknown
  source: /note=“Description of Unknown helical peptide”
KRLVQRLKDFLR

SEQ ID NO: 13 - protein, 12 aa, Unknown
  source: /note=“Description of Unknown helical peptide”
KRLIQRKRLIQR

SEQ ID NO: 14 - protein, 24 aa, Unknown
  source: /note=“Description of Unknown helical peptide”
LYKKLSKKLLGPGRLYKKLSKKLL

SEQ ID NO: 15 - protein, 26 aa, Unknown
  source: /note=“Description of Unknown helical peptide”
LIKLIKKILKKGPGRKKLIKKILKIL

SEQ ID NO: 16 - protein, 26 aa, Unknown
  source: /note=“Description of Unknown helical peptide”
LIRLIRRILRRGPGRRRLIRRILRIL

SEQ ID NO: 17 - protein, 26 aa, Unknown
  source: /note=“Description of Unknown helical peptide”
LIRLLRRILRRGPGRRRLIRRLLRIL

SEQ ID NO: 18 - protein, 26 aa, Unknown
  source: /note=“Description of Unknown helical peptide”
LIRLLREILRRGPGRERLIRRLLRIL

SEQ ID NO: 19 - protein, 26 aa, Unknown
  source: /note=“Description of Unknown helical peptide”
LIRLILRILRRGPGRRRLIRLILRIL

SEQ ID NO: 20 - protein, 26 aa, Unknown
  source: /note=“Description of Unknown helical peptide”
LIRLISRILRRGPGRRRLIRSILRIL

SEQ ID NO: 21 - protein, 27 aa, Unknown
  source: /note=“Description of Unknown helical peptide”
ALYLKDFKSSKSLDVSALADLKHLKRL

SEQ ID NO: 22 - protein, 28 aa, Unknown
  source: /note=“Description of Unknown helical peptide”
KRIVQRIKDFLRGPGRKRIVQRIKDFLR

SEQ ID NO: 23 - protein, 28 aa, Unknown
  source: /note=“Description of Unknown helical peptide”
KRLVQRLKDFLRGPGRKRLVQRLKDFLR

SEQ ID NO: 24 - protein, 28 aa, Unknown
  source: /note=“Description of Unknown helical peptide”
KRLIQRKRLIQRGPGRKRLIQRKRLIQR

SEQ ID NO: 25 - protein, 30 aa, Unknown
  source: /note=“Description of Unknown helical peptide”
LIKLIKKILKKGPGRKKLIKKILKILGPGR

SEQ ID NO: 26 - protein, 41 aa, Unknown
  source: /note=“Description of Unknown helical peptide”
KKLIKKILKILGPGRKKLIKEILKILGPGRKKLIKKILKIL

SEQ ID NO: 27 - protein, 58 aa, Unknown
  source: /note=“Description of Unknown helical peptide”
ALYLKDFKSSKSLDVSALADLKHLKRLGPGRALYLKDFKSSKSLDVSALADLKHLKRL

SEQ ID NO: 28 - protein, 30 aa, Unknown
  source: /note=“Description of Unknown helical peptide”
GRLIKLIKKILKKGPGRKKLIKKILKILGP

SEQ ID NO: 29 - protein, 26 aa, Unknown
  source: /note=“Description of Unknown helical peptide”
LIKLCKKILKKGPGRKKLIKKCLKIL

SEQ ID NO: 30 - protein, 11 aa, Unknown
  source: /note=“Description of Unknown helical peptide”
LIKKILKILKK

SEQ ID NO: 31 - protein, 11 aa, Unknown
  source: /note=“Description of Unknown helical peptide”
KKLAKEILKAL

SEQ ID NO: 32 - protein, 11 aa, Unknown
  source: /note=“Description of Unknown helical peptide”
KKLIKKILKIL

SEQ ID NO: 33 - protein, 11 aa, Unknown
  source: /note=“Description of Unknown helical peptide”
RRLIRRILRIL

SEQ ID NO: 34 - protein, 11 aa, Unknown
  source: /note=“Description of Unknown helical peptide”
RRLIRRILRIL

SEQ ID NO: 35 - protein, 26 aa, Unknown
  source: /note=“Description of Unknown helical peptide”
LIKLIKKILKKGPGRKKLIKKILKIL

SEQ ID NO: 36 - protein, 26 aa, Unknown
  source: /note=“Description of Unknown helical peptide”
LIRLIRRILRRGPGRRRLIRRILRIL

SEQ ID NO: 37 - protein, 26 aa, Unknown
  source: /note=“Description of Unknown helical peptide”
LIRLLRRILRRGPGRRRLIRRLLRIL

SEQ ID NO: 38 - protein, 28 aa, Unknown
  source: /note=“Description of Unknown helical peptide”
LLIKLIKKILKKGPGRKKLIKKILKILL

SEQ ID NO: 39 - protein, 28 aa, Unknown
  source: /note=“Description of Unknown helical peptide”
KRIVQRIKDFLRGPGRKRIVQRIKDFLR

SEQ ID NO: 40 - protein, 28 aa, Unknown
  source: /note=“Description of Unknown helical peptide”
KRLIQRKRLIQRGPGRKRLIQRKRLIQR

SEQ ID NO: 41 - protein, 28 aa, Unknown
  source: /note=“Description of Unknown helical peptide”
KLIKLIKKILKKGPGRKKLIKKILKILK

SEQ ID NO: 42 - protein, 28 aa, Unknown
  source: /note=“Description of Unknown helical peptide”
KLIRLIREILRRGPGRRRLIREILRILK

SEQ ID NO: 43 - protein, 28 aa, Unknown
  source: /note=“Description of Unknown helical peptide”
KEIVRRIKEFLRGPGRKEIVRRIKEFLR

SEQ ID NO: 44 - protein, 28 aa, Unknown
  source: /note=“Description of Unknown helical peptide”
KEIVRRIEKFLRGPGRKRIVERIEKFLR

SEQ ID NO: 45 - protein, 30 aa, Unknown
  source: /note=“Description of Unknown helical peptide”
HPLIKLIKKILKKGPGRKKLIKKILKILGH

SEQ ID NO: 46 - protein, 37 aa, Unknown
  source: /note=“Description of Unknown helical peptide”
ELLRRLLASLRRHDLLRGPGRELLRLLASLRRHDLLR

SEQ ID NO: 47 - protein, 40 aa, Unknown
  source: /note=“Description of Unknown helical peptide”
EALRSRLEKRIYILYRDTPVVKSSSRQREELLRISLRELE

SEQ ID NO: 48 - protein, 40 aa, Unknown
  source: /note=“Description of Unknown helical peptide”
RLLEKRLRRELERELRKQGPGRRLLEKRLRRELERELRKQ

SEQ ID NO: 49 - protein, 40 aa, Unknown
  source: /note=“Description of Unknown helical peptide”
RKQLRELIERLLERIRKLGPGRREQLERLIERLERLIEKR

SEQ ID NO: 50 - protein, 41 aa, Unknown; identical to SEQ ID NO: 26
  source: /note=“Description of Unknown helical peptide”

SEQ ID NO: 51 - protein, 30 aa, Unknown; identical to SEQ ID NO: 28
  source: /note=“Description of Unknown helical peptide”

SEQ ID NO: 52 - protein, 26 aa, Unknown
  source: /note=“Description of Unknown helical peptide”
LIKLCKKILKKGPGRKKLIKKCLKIL

SEQ ID NO: 53 - protein, 3 aa, Artificial Sequence
  source: /note=“Description of Artificial Sequence Synthetic peptide”
AAA

SEQ ID NO: 54 - protein, 10 aa, Artificial Sequence
  source: /note=“Description of Artificial Sequence Synthetic peptide”
GGGSSGGGSG

SEQ ID NO: 55 - protein, 4 aa, Artificial Sequence
  source: /note=“Description of Artificial Sequence Synthetic peptide”
GPGR

SEQ ID NO: 56 - protein, 8 aa, Artificial Sequence
  source: /note=“Description of Artificial Sequence Synthetic peptide”
RDTPVVKS

SEQ ID NO: 57 - protein, 27 aa, Artificial Sequence
  source: /note=“Description of Artificial Sequence Synthetic peptide”
AKDGIPAPTNYHKKHRAPVSCTGPAKM

SEQ ID NO: 58 - protein, 7 aa, Artificial Sequence
  source: /note=“Description of Artificial Sequence Synthetic peptide”
GSTAPPA

SEQ ID NO: 59 - protein, 25 aa, Artificial Sequence
  source: /note=“Description of Artificial Sequence Synthetic peptide”
RANATTLPKYYQNSRHPVSCTDPSK

SEQ ID NO: 60 - protein, 2 aa, Artificial Sequence
  source: /note=“Description of Artificial Sequence Synthetic peptide”
RW

SEQ ID NO: 61 - protein, 3 aa, Artificial Sequence
  source: /note=“Description of Artificial Sequence Synthetic peptide”
SRD

SEQ ID NO: 62 - protein, 7 aa, Artificial Sequence
  source: /note=“Description of Artificial Sequence Synthetic peptide”
GSTAPPA

SEQ ID NO: 63 - protein, 14 aa, Artificial Sequence
  source: /note=“Description of Artificial Sequence Synthetic peptide”
GSTAPPAGSTAPPA

SEQ ID NO: 64 - protein, 15 aa, Artificial Sequence
  source: /note=“Description of Artificial Sequence Synthetic peptide”
QASHTCVCEFNCAPL

SEQ ID NO: 65 - protein, 27 aa, Artificial Sequence
  source: /note=“Description of Artificial Sequence Synthetic peptide”
ARKKASIPNYYNSNLQPPVFCSDQSKM

SEQ ID NO: 66 - protein, 14 aa, Artificial Sequence
  source: /note=“Description of Artificial Sequence Synthetic peptide”
YEQGAGRGSTAPPA

SEQ ID NO: 67 - protein, 4 aa, Artificial Sequence
  source: /note=“Description of Artificial Sequence Synthetic peptide”
GSTA

SEQ ID NO: 68 - protein, 11 aa, Artificial Sequence
  source: /note=“Description of Artificial Sequence Synthetic peptide”
GGGSGGGTDGR

SEQ ID NO: 69 - protein, 20 aa, Unknown
  source: /note=“Description of Unknown helical peptide”
CRTRGCGCHLCRMLSQFTGG

SEQ ID NO: 70 - protein, 20 aa, Unknown
  source: /note=“Description of Unknown helical peptide”
CDMASSGCDVCECRVLLPEE

SEQ ID NO: 71 - protein, 25 aa, Unknown
  source: /note=“Description of Unknown helical peptide”
NQGRHFCGGALIHARFVMTAASCFQ

SEQ ID NO: 72 - protein, 25 aa, Unknown
  source: /note=“Description of Unknown helical peptide”
GDYLHFCMGALIHARTVMSGFDSDA

SEQ ID NO: 73 - protein, 18 aa, Unknown
  source: /note=“Description of Unknown helical peptide”
SIWRDWVDLICEFLSDWK

SEQ ID NO: 74 - protein, 32 aa, Unknown
  source: /note=“Description of Unknown helical peptide”
FTLKDKAKIWLNSLNPDSIRNWVDLQAEFLKK

SEQ ID NO: 75 - protein, 21 aa, Artificial Sequence
  source: /note=“Description of Artificial Sequence Synthetic peptide”
QFAFHGNSFPSGGFRLYLNAF

SEQ ID NO: 76 - protein, 15 aa, Artificial Sequence
  source: /note=“Description of Artificial Sequence Synthetic peptide”
GVNKKPQESLPHSRA

SEQ ID NO: 77 - protein, 15 aa, Artificial Sequence
  source: /note=“Description of Artificial Sequence Synthetic peptide”
ILDRFPTGTDPSVSD

SEQ ID NO: 78 - protein, 29 aa, Artificial Sequence
  source: /note=“Description of Artificial Sequence Synthetic peptide”
VMKKRSRQRKQRRRRRTTGRSSAIRRRAR

SEQ ID NO: 79 - protein, 34 aa, Artificial Sequence
  source: /note=“Description of Artificial Sequence Synthetic polypeptide”
DNSGSMIETLGGSGQFAFHGNSFPSGGFRLYLNA

SEQ ID NO: 80 - protein, 18 aa, Artificial Sequence
  source: /note=“Description of Artificial Sequence Synthetic peptide”
SIWRDWVDLICEFLSDWK

SEQ ID NO: 81 - protein, 32 aa, Vitis vinifera
FTLKDKAKIWLNSLRPRSIRNWVDLQAEFLKK

SEQ ID NO: 82 - protein, 19 aa, Artificial Sequence
  source: /note=“Description of Artificial Sequence Synthetic peptide”
GSWLRDIWDWICEVLSDFK

SEQ ID NO: 83 - protein, 20 aa, Vitis vinifera
DIWSWICELPNSDQWVESES

SEQ ID NO: 84 - protein, 18 aa, Artificial Sequence
  source: /note=“Description of Artificial Sequence Synthetic peptide”
DWLRIIWDWVCSVVSDFK

SEQ ID NO: 85 - protein, 18 aa, Vitis vinifera
RIIWNSWVPSTVSFFAWE

SEQ ID NO: 86 - protein, 18 aa, Artificial Sequence
  source: /note=“Description of Artificial Sequence Synthetic peptide”
SWLRDIWRWICKVLSRFK

SEQ ID NO: 87 - protein, 20 aa, Vitis vinifera
WLRLEKLEIWRICQMKCKVL

SEQ ID NO: 88 - protein, 18 aa, Artificial Sequence
  source: /note=“Description of Artificial Sequence Synthetic peptide”
KFDSLVECIWDWIDRLWS

SEQ ID NO: 89 - protein, 14 aa, Vitis vinifera
LVEAILDWIARMRS

SEQ ID NO: 90 - protein, 32 aa, Artificial Sequence; identical to SEQ ID NO: 74
  source: /note=“Description of Artificial Sequence Synthetic polypeptide”

SEQ ID NO: 91 - protein, 20 aa, Artificial Sequence
  source: /note=“Description of Artificial Sequence Synthetic peptide”
DIWSWICELPNSDQWVESES

SEQ ID NO: 92 - protein, 18 aa, Artificial Sequence
  source: /note=“Description of Artificial Sequence Synthetic peptide”
RIIWNSWDPSTVSFFAWE

SEQ ID NO: 93 - protein, 36 aa, Artificial Sequence
  source: /note=“Description of Artificial Sequence Synthetic polypeptide”
RLEKLEIWRICQMKCKGPGKRLEKLEIWRICQMKCK

SEQ ID NO: 94 - protein, 28 aa, Artificial Sequence
  source: /note=“Description of Artificial Sequence Synthetic peptide”
EAILDWIARMRSGPGKEAILDWIARMRS